(19) United States
(12) Patent Application Publication
(10) Pub. No.: US 2017/0354637 A1
(43) Pub. Date: Dec. 14, 2017

(54) FIBROTIC TISSUE STABILIZED BY CROSSLINKING FOR TREATMENT OF SNORING AND OBSTRUCTIVE SLEEP APNEA

(71) Applicant: Orthopeutics, LP, Lexington, KY (US)

(72) Inventors: Matthew E. Brown, Lexington, KY (US); Thomas P. Hedman, Lexington, KY (US)

(21) Appl. No.: 15/180,552

(22) Filed: Jun. 13, 2016

(51) Int. Cl. A61K 31/352; A61K 47/34; A61K 9/70; A61K 47/02; A61K 31/121; A61K 38/45; A61K 31/353; A61K 31/15; A61K 9/00

(52) U.S. Cl. CPC A61K 31/352; A61K 31/15; A61K 47/34; A61K 9/7007; A61K 9/0019; A61K 31/121; A61K 9/006; A61K 38/45; A61K 31/353; A61K 47/02; C12Y 203/02013

(57) ABSTRACT

The present invention relates to administration or application of a crosslink inducing agent to a subject following a stiffening procedure to the respiratory system. The present invention has identified that such application or administration of a crosslinking reagent to fibrotic tissue allows for improved stability and strength of the tissue.

### TECHNICAL FIELD

This invention relates to the treatment of sleep-related breathing disorders such as snoring and obstructive sleep apnea.

### BACKGROUND

Snoring is a common condition in the sleep disordered breathing spectrum (SDB) which also includes obstructive sleep apnea syndrome (OSAS) (Ioachimescu & Collop). It affects people of all ages but increases in likelihood with age (Demir et al., 2015). The reported prevalence of snoring within the US population varies with body mass index (BMI) between 25-70%, with the overall average among adults around 53% (Bhattacharyya, 2015). OSAS, a more severe SDB condition that occurs when the airway is substantially blocked while sleeping for short periods of time occurs in 2-24% of the population (Dyken & Bin Im, 2009; Ryan & Bradley, 2005; Young et al., 1993). The prevalence of OSAS in the general population is also a poorly diagnosed condition with approximately 70-80% of cases going undiagnosed (Ioachimescu & Collop). OSAS is also linked with increased likelihood of hypertension, cardiovascular disease, stroke, and motor vehicle accidents (Ferini-Strambi & Braghiroli, 2006; Hillman, Murphy, & Pezzullo, 2006; Sadatsafavi, Marra, Ayas, Stradling, & Fleetham, 2009).

Snoring and OSAS, while their etiology is not completely understood, are both the result of obstructed airflow in the airway. Snoring is a result of abnormal flow and excessive tissue compliance which causes a vibration of the soft tissue in the airways (primarily the soft palate) leading to the generation of sound (Counter & Wilson, 2004). In more severe cases, such as OSAS, the airway collapses and airflow is completely or substantially blocked for short periods of time which causes disruption of sleep (Jordan, McSharry, & Malhotra, 2014). These obstructions of airflow are typically caused by factors that affect upper airway (UA) collapsibility and those associated with anatomic narrowing of the airway. UA collapsibility is largely a function of tissue collapsing pressure, intraluminal pressure, and the compliance of the pharyngeal walls and structures (Ryan & Bradley, 2005). In most cases (56-75%), the upper airway collapse begins in the retropalatal-oropharyngeal area involving the soft palate (Ryan 2005, Suratt 1983). In addition, OSAS patients tend to have significant narrowing of the oropharyngeal, but not the naso- (superior to the soft palate) or hypopharyngeal (from the superior tip of the epiglottis to the larynx) airway regions in the supine position (Ingman, Nieminen, & Hurmerinta, 2004; Suratt, Dee, Atkinson, Armstrong, & Wilhoit, 1983). Consequently, even for the minority of patients where the initial site of the collapse is the hypopharynx, the collapse could well be attributed to the excessive negative airway pressure resulting from the airway narrowing and collapsibility in the region of the soft palate combined with the inspiratory activity of the diaphragm (Deegan & McNicholas, 1995). Anatomic narrowing of the airway is caused primarily by the natural structures of the surrounding tissues, deposited adipose tissue, craniofacial structure, enlarged tongues, and longer soft palates (Sutherland & Cistulli, 2015).

Mild to moderate snoring is typically treated first with lifestyle changes such as weight loss, avoiding alcohol before bed, and sleeping posture adjustment (Jordan et al., 2014; Kotecha & Hall, 2014). If the condition develops into OSAS the primary non-surgical treatments are continuous positive air pressure (CPAP) machines and mandibular advancement devices (MADs) (Spicuzza, Caruso, & Di Maria, 2015). CPAP machines are external devices worn while sleeping that prevent airway collapse by increasing the UA pressure which in turn decreases the number of obstructive events (Spicuzza et al., 2015). However, the efficacy of CPAP relies largely on patient compliance which has been reported to be between 60-70% (Jordan et al., 2014). MADs hold the mandible in a protruded position and keep the tongue away from the soft palate, which helps keep the airway open during sleep (Marcussen, Henriksen, & Thygesen, 2015). They can also increase the muscle tone in soft tissue of the mouth and throat as well as increase total upper airway volume which decreases the apnoea-hypopnea index (AHI) number (Marcussen et al., 2015).

A growing number of surgical interventions are being developed for patients who are CPAP intolerant or want a more permanent/less burdensome solution. Several of these procedures rely in some extent on the creation of scar tissue in the soft palate and tongue base (Kotecha & Hall, 2014). This scar tissue aims to increase the stiffness of the soft palate or tongue base to reduce the symptoms of snoring. However, scar tissue has been shown to be mechanically inferior to native tissue primarily due to its misaligned fiber orientation relative to native tissue (Evans, Oreffo, Healy, Thurner, & Man, 2013; Provenzano et al., 2003). The decrease in strength between the scar tissue or fibrotic tissue in general and native tissue can range from 50-70%, which can lead to mechanical failure (Hollinsky & Sandberg, 2007; Schmack et al., 2005). The case of inducing scarring on the soft palate or tongue base is likely complicated by the nightly exposure to mechanically destructive loading conditions while snoring, which could interrupt the healing process (Evans et al., 2013). The mechanical inferiority of scar tissue can also depend on the extent of healing—2.4% to 28% of normal strength according to the study by Schmack (2005). Chemicals such as sodium tetradecyl sulphate can be injected into the soft palate to create scar tissue and increase, at least temporarily, the stiffness of the tissue which can help alleviate the symptoms of snoring (Brietzke & Mair, 2001). Other agents that can be used to stiffen the soft palate include but are not limited to: ethanol, doxycycline, and hypertonic saline (Brietzke & Mair, 2004). One study reported that the success rate of these procedures dropped from 92% post treatment to 75% at 19 months and that there was an 18% snoring relapse rate (Brietzke & Mair, 2003). Radiofrequency ablation (RFA) or RF heating is another technique that involves the creation of scar tissue in the palate region in order to stiffen the palate and reduce snoring (Back, Hytonen, Roine, & Malmivaara, 2009). Initial results are promising however long term (12-16 months) results show only a 50% success rate (Neruntarat & Chantapant, 2009). The Pillar procedure involves the implantation of palatal rods in the soft palate with the intention of creating scar tissue to stiffen the soft palate (Ho, Wei, & Chung, 2004). These implants showed moderately successful initial results with approximately 80% of patients reporting snoring improvements but long term results are poor, based on anecdotal evidence and unpublished clinical observations. Long-term data is currently unavailable and is likely similar to other palatal stiffening techniques that rely on tissue microstructure that is mechanically inferior to healthy native tissues (Ho et al., 2004).

International Published Patent Application WO2001070151 A1 to Aksan et al. discloses a method of shrinking collagen within joints to increase joint stability followed quickly by crosslinking to maintain the tissue in this state and prevent relaxation/stretching over time. During the shrinking process, collagen is denatured using heat, radiofrequency, ultrasound, magnetic fields, microwaves, or a heated solution. During denaturation, the collagen shrinks which can act to stabilize joints with acquired joint laxity. This shrinkage effect is then made more permanent by crosslinking the newly denatured tissue before the mechanical stresses of the joint can stretch it back out.

Inflammation is the first stage of wound healing and also involves the laying down of granulation tissue which helps prevent foreign body invasion and provides new vasculature (Evans et al., 2013). The next stage is tissue formation in which the extracellular matrix (ECM) that was quickly formed is remodeled into a collagenous matrix that can form a fibrous scar tissue (Evans et al., 2013). Over time the body will attempt to replace the fibrous scar tissue with new native tissue, in sufficiently deep or large wounds this is not possible and what is left is scar tissue (Evans et al., 2013). It is preferentially during the peak of this fibrous tissue formation that the present invention described herein applies a crosslinker to strengthen the tissue in and around the fibrotic tissue region, reducing tissue compliance, and preventing the scar tissue from being resorbed and remodeled by the body (likely between days-3 and 10 post scar tissue creation) (Gurtner, Werner, Barrandon, & Longaker, 2008).

Advantages of the palate stiffening techniques described above include being minimally invasive, having a low mortality rate, and patient convenience/compliance. The one major drawback that all the existing stiffening techniques regardless of method share is the lack of long term effectiveness. This can likely be attributed to the fundamental mechanism by which they achieve palate stiffening, the creation of fibrotic or scar tissue. In addition to the known mechanical inferiority of fibrotic tissue expressed above, the role of scar tissue is to be temporary and it is expected to soften and be remodeled by the body over time, causing the treatment to lose effectiveness as the body heals itself from the damage (Courey et al., 1999).

In addition to crosslinking newly formed scar or fibrotic tissue in the soft palate or tongue base, the present invention can be applied to other conditions in which scar tissue is often the point of failure. Hernia repairs have a 31-49% recurrence rate for open suture repair and a 0-10% recurrence rate for open mesh repair (Cassar & Munro, 2002). Newer techniques such as laparoscopic incisional repair still have recurrence rates between 0-9% with technique being cited as a large factor (Cassar & Munro, 2002). Pelvic organ prolapse is a condition where the pelvic organs descend downward and can result in a protrusion of the vagina, uterus, or both organs (Jelovsek, Maher, & Barber, 2007). The condition affects 43-76% of women seeking routine gynecological care and 3-6% are considered clinically significant (Jelovsek et al., 2007). Patients who have a surgical procedure done also have to have a repeat operation 13% of the time (Jelovsek et al., 2007). Application of a crosslinker to the incisional scar and supplemental closure devices as described herein may then provide for increased wound stability and reduced recurrence rates.

### SUMMARY

In accordance herein, the present invention provides a method of treatment for flaccid soft palate or other airway tissues, wherein fibrotic or scar tissue is created to stiffen or otherwise change the mechanical properties of the upper airway tissues, and the fibrotic or scar tissue and adjacent tissues are stabilized mechanically and enzymatically via application of a chemical crosslinker applied to the fibrotic or scarred tissue and adjacent tissues. The crosslinker can be applied to the tissue via an injection, spraying, or brushing on of a crosslinker reagent, or via release from a degradable material, or via another time-dependent release method. This crosslinking can reinforce the temporary scar tissue, improving its mechanical properties, reducing tissue compliance, reducing propensity for tissue collapse, reducing vibration amplitudes, and allowing the fibrotic or scar tissue to remain in the surrounding tissue for longer periods of time, lengthening the lifespan of the snore or apnea reducing treatment.

The present invention described herein provides a process that avoids denatured collagen or shrinkage of collagenous tissues. The present invention targets newly formed fibrotic/scar tissues and the adjacent tissues. The present invention follows or accompanies the body's induced fibrosis or wound healing process which creates fibrotic or scar tissue.

Thus, in one aspect of the invention, a follow-up treatment for snoring and/or OSAS is provided which includes the crosslinking of the soft palate or other upper airway tissues, using protein crosslinking reagents, which have had fibrosis induced for the purpose of stiffening the tissue. The treatment stabilizes the newly created fibrosis or scar tissue in order to improve and increase the longevity of the effectiveness of the therapy.

In another aspect of the invention, crosslinking of the tongue base of a subject or patient is provided in order to stabilize the induced fibrosis and maintain the stiffening effects for long-term treatment of snoring and OSAS.

In another aspect of the invention, the crosslinking of the fibrotic or scar tissue and surrounding native tissues is provided in order to further increase tissue stiffness, or further reduce tissue compliance, or further reduce propensity for tissue collapse, or further reduce vibration amplitudes, or further increase mechanical support, or stabilize the fibrotic or scar and surrounding tissues or region of tissues.

In a further aspect of the invention, methods and associated devices for modifying fibrosis/scar tissue inducing techniques by application of a protein crosslinker or multiple crosslinkers to allow for longer effective treatment times and/or increased treatment effects is provided. The fibrosis inducing techniques can be applied to either the soft palate or the tongue base of a subject. The fibrosus inducing techniques include those done with application of radio-frequency (RF) heating, RFA, ethanol, hypertonic saline, sodium tetradecyl sulphate, doxycycline, palatal implants, electrical, light, or gas based. Also, in any embodiment, the crosslinker can be delivered to the tissue via injection, spraying, brushing, a patch, release from a device, release from a coating on a device, release from a gel, encapsulation in a liquid or biomaterial, a rapidly dissolving thin strip-type delivery device, a suture, palatal implants, or any combination thereof.

In another aspect of the invention, the submucosal or sub surface tissue of the soft palate is the targeted region of the fibrotic or scar and surrounding tissues to be treated with the protein crosslinking agent. Consequently methods and associated devices for treating fibrotic or scar and surrounding tissues provide for the delivery to the sub-surface region of the tissue. In the case of delivery of the crosslinking agent via an injection, a needle tip is directed to the submucosal tissues and remains in this position for the duration of the injection. In the case of an agent releasing device or coating on a device such as a suture, the device is preferentially implanted into the submucosal region of the tissue. In the case of delivery via a patch applied to the surface of the soft palate, the patch contains microneedles for preferential delivery of the crosslinker to the subsurface region of the tissue.

In another aspect of the invention, methods and associated devices provide for modifying fibrotic or scar tissue in other soft tissues or connective tissues by the application of a protein crosslinker or crosslinkers to allow for longer tissue stabilization, reduction of tissue compliance and improvement of structural support. In any embodiment, the crosslinker can be delivered to the tissue via injection, spraying, brushing, a patch, an agent releasing device, an agent releasing coating on a device, a gel, encapsulation, a strip-type delivery device, a suture, palatal implants, or any combination thereof.

### DETAILED DESCRIPTION

The present invention provides for administration or application of a crosslinker to fibrotic tissue. The crosslinker may be administered to fibrotic tissue within a subject through multiple means, either alone or in combination. The crosslinker may be applied or administered as a solution, spray, gel, encapsulation, device, coating, patch, strip, suture, implant or combination thereof.

Protein crosslinking has been previously used to modify the mechanical properties and chemical stability of collagenous tissues (Charulatha & Rajaram, 2003; Chuang, Odono, & Hedman, 2007; Han, Jaurequi, Tang, & Nimni, 2003; Slusarewicz, Zhu, & Hedman, 2010; Sung, Liang, Chen, Huang, & Liang, 2001; Tang, Sharan, & Vashishth, 2008; Vasudev & Chandy, 1997; Zhai et al., 2006). This has typically been done ex vivo to modulate the strength of tissue implants as well as make them more resistant to enzymatic degradation (Hapach, VanderBurgh, Miller, & Reinhart-King, 2015). Hedman, et al., proposed the use of minimally toxic chemical crosslinkers to treat native tissues in vivo (Chuang, Odono, & Hedman, 2007; Slusarewicz, Zhu, & Hedman, 2010; Slusarewicz, Zhu, Kirking, Toungate, & Hedman, 2011). These prior chemical crosslinking techniques were directed at well-organized but mechanically insufficient or mechanically degraded native tissues, not fibrotic or scar tissues.

Lau et al. investigated a biological, gene activated matrix approach to generate overexpression of lysyl oxidase to increase crosslinking of collagen and elastin in wound healing applications (Lau, Gobin, & West, 2006). Lysyl oxidase is a native protein that is involved in the reorganization of collagen fibers in granulation tissue by way of inducing directional migration of fibroblasts and catalyzing inter- and intra-molecular covalent crosslinks. In this way the native protein LO is involved in the formation of the fibrotic tissues associated with wound healing and with the remodeling of these tissues. In contrast, a chemical crosslinking approach would act in a non-biologic way to increase the strength and stiffening of the fibrotic or scar tissues and prevent remodeling of these tissues. Essentially chemical crosslinking of fibrotic or scar tissues halts the wound healing process, while augmenting the mechanical properties in such a way as to reduce tissue collapsibility and excessive compliance, whereas LO overexpression promotes the complete wound healing process. In the case of the use of induced fibrosis to stiffen upper airway tissues, the full remodeling process leads eventually to a loss of the mechanical property changes that were the target of the induced fibrosis procedure. And, as has been expressed above, without the timely application of a crosslinking treatment, the beneficial changes brought about by the induced fibrosis are lost to natural remodeling of the fibrotic or scar tissues, even without the overexpression of LO.

To be an effective in vivo crosslinker, the applied chemical would need to be non-toxic or minimally toxic and react quickly to avoid clearance before action. Such possible chemical crosslinking agents include but are not limited to D- or L-Threose, genipin (GP), methylglyoxal (MG), 1-ethyl-3-(3-dimethylamniopropyl) carbodiimide hydrochloride (EDC), proanthrocyanidin, and transglutaminase (TG). Different crosslinkers have been shown to elicit different mechanical effects on tissues (Slusarewicz et al., 2011) and thus different crosslinkers may be used alone or in combination to modulate the final mechanical properties of the fibrotic tissue or fibrotic and surrounding tissues. Thus the applied solutions, sprays, gels, encapsulations, devices, coatings, patches, strips, sutures, or implants of the present invention may contain at least one chemical crosslinker or a mixture of two or more crosslinking agents. The crosslinkers can be administered in an amount specific to achieve the desired effect and can be administered in a single or multiple administrations. For example, MG can be administered at between about 5 to 50 mM, PA can be administered at between about 0.025 to 0.5% w/v, EDC can be administered at between about 2 to 50 mM, and DT or LT can be administered at between about 20 and 100 mM. Further examples of crosslinkers and their buffers and concentrations can be found in U.S. Pat. No. 8,283,322, which is hereby incorporated by reference in its entirety.

In the various aspects of the invention, the crosslinker treatment may be applied to a subject at different time points post-surgery, dependent on timeframe for fibrotic tissue formation, preferably within 3 days to 3 weeks but ideally 3-10 days post-surgery.

In one aspect of the present invention, a solution of crosslinking reagent, in a suitable carrier solution, is injected into the soft palate or tongue base after a stiffening procedure is performed and fibrotic tissue is in place. The nature of methods and devices leading to fibrotic tissue formation can be implant, electrical, light, gas, or chemical based, summarized in Kotecha and Hall (2014) and elsewhere. Chemicals such as sodium tetradecyl sulphate can be injected into the soft palate to create scar tissue and increase, at least temporarily, the stiffness of the tissue which can help alleviate the symptoms of snoring (Brietzke & Mair, 2001). Other agents that can be used to stiffen the soft palate include but are not limited to: ethanol, doxycycline, and hypertonic saline (Brietzke & Mair, 2003, 2004). Radiofrequency ablation (RFA), use of lasers, or RF non-ablative heating are other techniques that involves the creation of scar tissue in the palate region in order to stiffen the palate and reduce snoring (Back, Hytonen, Roine, & Malmivaara, 2009, Neruntarat & Chantapant, 2009). The Pillar procedure involves the implantation of palatal rods in the soft palate with the intention of both augmenting the stiffness of the soft palate by the stiffness of the implants and by the implants and implantation surgery creating scar tissue to stiffen the soft palate (Ho, Wei, & Chung, 2004). As the protein crosslinker reacts with the fibrotic tissue, it can induce crosslink formation that increases the mechanical strength and reduces compliance of the fibrotic and surrounding tissues, and stabilizes the fibrotic or scar tissue and slows resorption and enzymatic degradation. The cros slink augmentation of the present invention can also increase the fibrotic tissue's resistance to subsequent mechanical degradation, which is expected to occur given its poorly organized nature and relatively low strength. The crosslinking reagent can be applied, such by injection, in one location or multiple locations in or around the scar tissue to facilitate a desired distribution of agent or agents and stabilization of fibrotic or scar tissue and surrounding native tissue.

The carrier solution for the crosslinking reagent can be aqueous or non-aqueous and may contain other non-crosslinking components that may help facilitate crosslinking. Such components include, but are not limited to, buffers (in order to maintain a pH that is optimal to accelerate or extend the crosslinking activity for a particular crosslinker), surfactants (to enhance the distribution of the crosslinker within the tissue), stabilization agents (to maintain the stability of the solution), and co-factors (to enhance the reactivity of the crosslinker). The addition of a flavoring agent may also be used to make the oral treatment more tolerable to patients.

The crosslinker may be administered to the subject or patient once or over a series of treatments. The crosslinker can be selected from a number of minimally toxic crosslinking agents such as genipin at concentrations between 5-120 mM (preferably about 20-100 mM). Likewise, the buffer can be selected from a number of solutions such as sterile water for injection (with or without a solute to adjust osmolarity), or sterile saline solution, or at a concentration of 25-250 mM 4-(2-Hydroxyethyl)-1-piperazinepropanesulfonic acid (EPPS) (preferably approximately 50 mM) at a pH between 7-10 (preferably about 7.4). The crosslinker reagent can optionally contain 25-250 mM of a phosphate salt (preferably 50 mM) to act as a co-factor and to adjust osmolarity and also optionally contain a co-solvent such as dimethyl sulfoxide (DMSO) in a range between 1-50% (preferably between 10-20%) to increase the solubility of the crosslinking agent.

In some embodiments, the crosslinker can be methylglyoxal at a concentration ranging from 10-60 mM in sterile water for injection, or in sterile saline solution, or in about 50 mM EPPS buffer, at a pH of about 7.4, and with a solute such as phosphate to adjust osmolarity of the solution.

In some embodiments, the crosslinker can be proanthrocyanidin at a concentration ranging from 0.025-0.5% w/v in sterile water for injection, or in sterile saline solution, or in about 50 mM EPPS buffer, at a pH of about 7.4, and with a solute such as phosphate to adjust osmolarity of the solution.

In some embodiments, the crosslinker can be 1-ethyl-3-(3-dimethylamniopropyl) carbodiimide hydrochloride at a concentration ranging from 2-50 mM in about 50 mM 2-(N-morpholino)ethanesulfonic acid (MES) buffer, at a pH of about 6.0.

In some embodiments, the crosslinker can be L- or D-threose at a concentration ranging from 20-100 mM in sterile water for injection, or in sterile saline solution, or in about 50 mM EPPS buffer, at a pH of about 7.4, and with a solute such as phosphate to adjust osmolarity of the solution.

In some embodiments, the crosslinker can be transglutaminase at a concentration ranging from 0.5-5 U/ml in sterile water for injection, or in sterile saline solution, or in about 50 mM Tris buffer, at a pH of about 7.4, and with a solute such as phosphate to adjust osmolarity of the solution.

In another aspect of the present invention, the crosslinking solution described above is administered to the patient in the form of an aerosol spray at crosslinker concentrations and in a buffered carrier similar to that described above. The crosslinking agent can diffuse into the fibrotic tissue in order to stabilize it, improve the mechanical properties, and prevent resorption/degradation. The crosslinker can be delivered from a pump that is pressurized with a suitable gaseous or liquid propellant or one that is actuated using a hand or motorized pump. In this embodiment, the crosslinker could be in the form of lyophilized, sterile tablet or cake that is placed in an aerosol pump containing the carrier solution and shaken until fully solubilized in the solution. One advantage to this delivery method is that it is non-invasive. In addition, the formulation may contain both thickening agents and biocompatible adhesives in order to coat the fibrotic tissue and maintain contact for a prolonged period of time while the crosslinking takes place. The formulation may also contain a surfactant or penetrant to enhance penetration from the surface of the tissue to the underlying tissue. Suitable thickening agents might include, but are not limited to, gellan gum, alginates, agar, carrageenan and pectin, proteins such as gelatin and artificial molecules such as Carbomer (polyacrylic acid), and polyethylene glycol. Non-limiting examples of adhesives might include poly(glycerol-co-sebacate acrylate), oleic methyl esters, or alkyl ester cyanoacrylates. An example of a penetration enhancer includes dimethyl sulfoxide (DMSO).

In a further aspect of the present invention, the crosslinking agent can be delivered via a patch or a group of patches that is placed on the surface of the fibrotic tissue. The crosslinker can be incorporated into the patch or into a delivery vehicle that coats the surface of the patch that is in contact with the fibrotic tissue. In order to maintain contact with the tissue, the patch may be attached using biocompatible adhesives (such as those described above), biodegradable or semi-permanent sutures, or a tack or staple like device. The patch may contain microneedles for delivery of the crosslinker to the submucosal tissues. The delivery vehicle may be either a suitable solvent, or solution containing appropriate excipients described with or without suitable thickening agents. The formulation may also optionally contain penetration enhancing reagents such as DMSO in order to facilitate the entry of the crosslinker into the tissue. Additionally the formulation can also contain flavoring agents to make the patch more palatable in the patient's mouth. Alternatively the solution may be incorporated into the patch material. This crosslinker may also be in a solid form which would dissolve slowly over time once in contact with bodily fluids, thereby providing a delayed and sustained release of crosslinker over time. In the case of non-degradable polymers, the material of the patch should be porous and the patch would be later removed by a clinician once its function has been completed. In the case of biocompatible non-toxic polymers (such as polylactic or polyglycolic acid), the patch degradation products may be swallowed slowly and cleared by the body as the patch broke down. The crosslinking agent in such an instance should be incorporated into the patch and released to diffuse into the tissue as the patch degrades on the tissue over time. In this embodiment, a patch would not have to be removed by a clinician. In the cases of degradable or non-degradable materials, the patches may be comprised of or coated with a layer or multiple layers of biodegradable polymers that may or may not contain crosslinking agent. Such may facilitate sustained release (for instance in the case of multiple crosslinker containing layers) or delayed release (for instance in the case of a non-crosslinker containing outer layer surrounding a crosslinker containing patch). Different crosslinkers can also be used in different layers of a patch to control the release rates of particular agents over time. These layers may also be used to deliver a flavoring agent to make the patch a more palatable device to be in the mouth while the crosslinking is taking place. In all cases, the patch could be designed to allow airflow in the case of accidental dislodging from the target tissue. This may be achieved using a number of techniques such as; a pattern of perforations and/or intersecting cut-lines that would enable portions of the patch to deform if not attached to the tissue, holes to facilitate airflow, a combination of flaps and holes, by being small enough to not fully obstruct airflow (for instance in the case of a group of smaller patches), or being shaped in such a way that makes blockage unlikely.

Solid or liquid crosslinkers can be incorporated into a patch by addition of the crosslinker to a molten polymer prior to casting, molding, spinning, or other manufacturing process. Alternatively, the crosslinker can be co-solubilized with the polymer in a suitable solvent (for example, acetone) and then incorporated into the device, such as by solvent evaporation or by precipitation (for example, by the addition of ethanol) of the polymer as described previously (Athanasiou, Singhal, Agrawal, & Boyan, 1995; Singhal, Agrawal, & Athanasiou, 1996). The crosslinker and polymer can also be solubilized separately and mixed prior to precipitation in either the same solvent or different (miscible) solvents. The rate of crosslinker release from the patch can be controlled by varying the concentration of crosslinker as well as by selecting polymer materials with different in vivo degradation profiles.

The degradation of biodegradable polymers is often associated with a decrease in local pH due to the production of acidic monomers which can negatively affect the efficiency of many protein crosslinkers (Slusarewicz et al., 2010). The acidification of the local region can be counter balanced with the addition of basic salts into the polymer matrix or crosslinker solution/solid (Agrawal & Athanasiou, 1997). Such salts may be inorganic (for example, but not limited to, calcium carbonate, calcium hydroxyapatite, or sodium bicarbonate) or organic (for example, but not limited to, 2-amino-2-hydroxymethyl-propane-,3-diol (Tris) or 4-(2-hydroxyethyl)-1-piperazineethanesulfonic acid (HEPES)). These basic salts can be incorporated at a level of about 5-50% by weight of the polymer (preferably 15-30%).

In another aspect of the present invention, the crosslinker is incorporated into a non-degradable strip-type delivery device, such as those used in a Pillar procedure of the soft palate (Ho, et al. 2004). The crosslinker may be incorporated in any of the ways described herein. The crosslinker may be distributed throughout the non-degradable polymer at a ratio of about 5-100 mg crosslinker to 1 g of polymer. In some embodiments, the crosslinker may be genipin and the genipin may be distributed throughout the non-degradable polymer at a ratio of about 10 mg genipin to 1 g of polymer. Following implantation, the crosslinker can diffuse into and crosslink the fibrous tissue in order to enhance and stabilize the stiffening effects resulting from the induced fibrosis. The crosslinking can help reduce the resorption and mechanical degradation that occurs over time to the unorganized and otherwise mechanically inferior fibrotic or scar tissue.

In addition, the polymer of the strip may be constructed using a biodegradable polymer, which would allow for a delayed release of the crosslinker after sufficient time for scar formation has been allowed before the strip degrades. The crosslinker can be any of several known minimally toxic crosslinking agents such as genipin or methylglyoxal (preferably about 1-20 mg per strip, although more can be incorporated if needed). The crosslinker may be distributed throughout the biodegradable polymer at a ratio of about 5-100 mg crosslinker to 1 g of polymer. In some embodiments, the crosslinker may be genipin and the genipin may be distributed throughout the biodegradable polymer at a ratio of about 10 mg genipin to 1 g of polymer.

In another aspect of the invention, the degradable or non-degradable crosslinker delivery device is a suture, or sphere, or pellet, or a number of these devices that is/are inserted into the submucosal region of the tissue. In instances where the device is a stiffening and crosslinker delivery device, the device(s) may be inserted at the time of or after the fibrosis is induced by any method described herein. In the instances that the device is also a means by which the fibrosis is to be generated (as indicated herein), then the time of insertion will determine the onset of fibrosis generation. The delivery device or devices are positioned in the tissue in such a way as to provide crosslinking treatment to the surrounding fibrotic or scar tissue and surrounding intact tissues upon diffusion of the crosslinking agent from the device into the tissue. In another aspect, the device of any type is coated with a crosslinking agent loaded degradable or non-degradable coating and the crosslinker is released over time into the surrounding fibrotic or scar or surrounding intact tissues. In another aspect, the device of any type or geometry which is inserted into the target region of the target tissue is the means by which fibrosis is induced in the targeted tissue, and the same device or a coating on the device contains the crosslinking agent or agents that are released in a delayed fashion so as to form crosslinks in the recently formed fibrotic tissues and surrounding tissues. In another aspect, the delivery device or devices of any type contains the crosslinking agent or agents, and the same device is coated with an outer biodegradable coating that does not contain a crosslinking agent but prevents release of the crosslinker from the underlying device or coating until the outer coating has sufficiently degraded.

In another aspect of the invention, the polymer coating on strip-type devices used to induce fibrosis would only degrade and release crosslinker after certain environmental conditions are met. This may take the form of a polymer coating that begins degrading around pH 7.4 (normal body pH) and doesn't degrade in the lowered pH of the tissue undergoing wound healing. Alternatively, the crosslinker may only be released from the strip device when certain cytokines are detected or the pH becomes even more acidic with the combination of wound healing and acidic polymeric degradation products (such as lactic and glycolic acid). The biodegradable polymer coating on the strip-type device may also degrade in the presence of end stage wound healing enzymes which function primarily to resorb fibrotic tissue for replacement. Some examples of pH sensitive polymers include, but are not limited to, poly(carboxylic acids), methacrylic acid, methyl methacrylate, and cellulose derived polymers.

The crosslinker delivery vehicles described herein may be single phase systems consisting of a solution of crosslinking agent either in a solvent or a solvent containing excipients and/or thickening agents. In other aspects of the invention, the carrier vehicle is composed of a two phase system such as an oil in water (O/W) or water in oil (W/O) emulsion. The emulsion may also contain three or more phases such as a water in oil in water (W/O/W) emulsion with the crosslinker incorporated into one or more of the phases as needed. The formulations herein are not to be considered limited to simple emulsion systems and may also include more complex systems such as liposomal formulations and multi-vesicular emulsions.

In another aspect of the invention, the delivery vehicle is composed of polymeric (degradable or non-degradable) micro or nanospheres which can encapsulate the crosslinking agent. The spheres may serve as a reservoir for the crosslinking agent and release it into the fibrotic tissue as they are degraded or swelled with water.

Those skilled in the art will appreciate that the claimed invention further comprises compositions and kits for crosslinking fibrotic tissue. Compositions may comprise, for example, a crosslinker as described herein and a carrier solution. The carrier solution may include a buffer, a surfactant, a stabilization agent, a co-factor, a flavoring agent or combinations thereof. The composition may further comprise additives to assist in controlling or delaying the release of the crosslinker, such as biodegradable polymers. Kits may similarly comprise a crosslinker in solution or powder/solid form and optionally a solution to thereafter dissolve in, and a carrier solution. Each component may be separate within the kit, such as in a sterile, isolated environment, such as a vial. The kit may further include directions as to preparing the chemical crosslinking reagent and to how to administer as described herein.

The present invention may be better understood by referring to the accompanying examples, which are intended for illustration purposes only and should not in any sense be construed as limiting the scope of the invention.

### Example 1

A practitioner can treat a patient who has already had a soft palate stiffening procedure to create fibrotic tissue by a follow-up injection into the soft palate of 50 mM genipin in a buffer solution of sterile water for injection with about 100-120 mM tri-sodium phosphate. The follow-up crosslinker injection can occur 3-10 days following the soft palate stiffening, fibrosis inducing procedure. Sterile water for injection can be injected into a vial containing the sterile genipin and tri-sodium phosphate and then the vial is shaken for 5 minutes or until the solid components are completely solubilized. The injection procedure can be performed with direct observation using smaller volumes injected into multiple sub-mucosal locations in the soft palate.

### Example 2

A practitioner can treat patients who have had a palate stiffening procedure done by applying a crosslinker such as genipin to the fibrotic tissue to stabilize it in the form of an aerosol spray. This crosslinking treatment can be applied 3-10 days following the hard palate stiffening, fibrosis inducing procedure. This genipin spray could be delivered from either a self-containing pressurized container or an actuated pump. The propellant such as a chlorofluorocarbon and the crosslinker can be contained in separate compartments or vessels and the components mixed prior to pressurization or delivery. In the case of an actuated pump, the propellant can be sterile water and the sterile crosslinker in the form of a lyophilized cake can be added to the propellant prior to spraying. The propellant can also contain a thickening agent such as gellan gum, and a biocompatible adhesive such as poly(glycerol-co-sebacate acrylate), and a penetration enhancer such as DMSO.

### Example 3

A practitioner can treat patients who have had a palate stiffening procedure done by applying a patch composed of a non-degradable polymer, polyurethane, that sticks to the fibrotic tissue while releasing crosslinker via microneedles that penetrate the mucosal layer of the soft palate. The patch can be applied 3-10 days following the hard palate stiffening, fibrosis inducing procedure. The patch would need to be removed after a set period of time by either the patient or a trained healthcare professional. The patch is constructed by dispersing solid genipin in the liquid polymer during manufacture at a ratio of 10 mg genipin to 1 g polymer.

### Example 4

A practitioner can treat patients who have had a palate stiffening procedure done by applying a patch composed of a biodegradable polymer comprised of poly (lactic acid) that sticks to the fibrotic tissue while degrading and releasing crosslinker. The patch can be applied 3-10 days following the hard palate stiffening, fibrosis inducing procedure. No removal of the patch would be necessary as the material would slowly degrade as it released crosslinker and the degradation products would be swallowed and cleared by the patient's body. The patch is constructed by dispersing solid genipin in the liquid polymer during manufacture at a ratio of 10 mg genipin to 1 g polymer. The tissue contacting surface of the patch can contain a biocompatible adhesive such as poly(glycerol-co-sebacate acrylate).

### Example 5

A practitioner can treat patients who plan to have a palate stiffening procedure done, similar to the Pillar Procedure, where non-degradable polymer strips are implanted to induce scar tissue formation. These non-degradable polymer strips can be coated in a degradable polymer, such as poly(lactic or poly(glycolic acid), that contains approximately 10 mg of genipin to be released into the tissue as the polymer degrades. The polymer strips can be coated by either immersing them in molten coating material containing the crosslinking agent or by repeated immersions in a polymer-solvent-crosslinker solution in which the solvent could be evaporated off leaving only the coating material and crosslinker on the strips.

### Example 6

A practitioner can treat patients who have had a palate stiffening procedure done by inserting genipin coated suture(s) into the fibrotic tissue in order to crosslink it. The sutures with genipin loaded coatings can be inserted 3-10 days following the hard palate stiffening, fibrosis inducing procedure. The coating can be put on the suture by immersing the suture in a polymer (poly(lactic acid or poly(glycolic acid)) that has been solubilized in a solvent along with the crosslinking agent genipin at a concentration around 50 mM. The solvent can be evaporated off leaving only the coating and genipin on the suture. These sutures could be degradable like the coating such that no second procedure is required to retrieve the delivery vehicle (suture/suture coating).

### Example 7

A practitioner can treat patients by inserting genipin coated suture(s) into the fibrotic tissue in order to both induce a fibrotic response and to crosslink the fibrotic tissue. The coating can be put on the suture by immersing the suture in a polymer (poly(lactic acid or poly(glycolic acid)) that has been solubilized in a solvent along with the crosslinking agent genipin at a concentration around 50 mM. The solvent can be evaporated off leaving only the coating and genipin on the suture. These sutures could be degradable like the coating such that no second procedure is required to retrieve the delivery vehicle (suture/suture coating).

### Example 8

A practitioner can treat patients who have had a tongue base stiffening procedure done by injecting or spraying genipin at a concentration around 50 mM in order to stabilize the fibrotic tissue and retain its beneficial effects for a longer period of time. The injection or spraying can be performed 3-10 days following the hard palate stiffening, fibrosis inducing procedure. The addition of about 50-100 mM phosphate solution can be added to the genipin in the case of an injection in order to increase the amount of crosslinking. In the case of an aerosol spray, a propellant such as a chlorofluorocarbon can be included with or without 50-100 mM phosphate to facilitate delivery and crosslinking in the target fibrotic tissue.

The foregoing examples have been presented for purposes of illustration and description. They are not intended to be exhaustive or to limit the embodiments to the precise forms disclosed. Obvious modifications and variations are possible in light of the above teachings. All such modifications and variations are within the scope of the appended claims when interpreted in accordance with the breadth to which they are fairly, legally and equitably entitled. All references to patent related and non-patent related literature are hereby incorporated by reference in their entirety.

### REFERENCES

Agrawal, C. M., & Athanasiou, K. A. (1997). Technique to control pH in vicinity of biodegrading PLA-PGA implants. Journal of Biomedical Materials Research, 38(2), 105-114. doi:10.1002/(sici)1097-4636(199722)38:2<105::aid-jbm4>3.0.co;2-u
Athanasiou, K. A., Singhal, A. R., Agrawal, C. M., & Boyan, B. D. (1995). IN-VITRO DEGRADATION AND RELEASE CHARACTERISTICS OF BIODEGRADABLE IMPLANTS CONTAINING TRYPSIN-INHIBITOR. Clinical orthopaedics and related research(315), 272-281. Retrieved from <Go to ISI>://WOS:A1995RC03500033
Back, L. J. J., Hytonen, M. L., Roine, R. P., & Malmivaara, A. O. V. (2009). Radiofrequency Ablation Treatment of Soft Palate for Patients with Snoring: A Systematic Review of Effectiveness and Adverse Effects. Laryngoscope, 119(6), 1241-1250. doi:10.1002/lary.20215
Bhattacharyya, N. (2015). Sleep and Health Implications of Snoring: A Populational Analysis. Laryngoscope, 125(10), 2413-2416. doi:10.1002/lary.25346
Brietzke, S. E., & Mair, E. A. (2001). Injection snoreplasty: How to treat snoring without all the pain and expense. Otolaryngology-Head and Neck Surgery, 124(5), 503-510. doi:10.1067/mhn.2001.115400
Brietzke, S. E., & Mair, E. A. (2003). Injection snoreplasty: Extended follow-up and new objective data. Otolaryngology-Head and Neck Surgery, 128(5), 605-615. doi:10.1016/s0197-5998(03)00229-8
Brietzke, S. E., & Mair, E. A. (2004). Injection snoreplasty: Investigation of alternative sclerotherapy agents. Otolaryngology-Head and Neck Surgery, 130(1), 47-57. doi:10.1016/j.otohns.2003.08.004
Cassar, K., & Munro, A. (2002). Surgical treatment of incisional hernia. British Journal of Surgery, 89(5), 534-545. doi:10.1046/j.1365-2168.2002.02083.x
Charulatha, V., & Rajaram, A. (2003). Influence of different crosslinking treatments on the physical properties of collagen membranes. Biomaterials, 24(5), 759-767. doi:10.1016/s0142-9612(02)00412-x
Chuang, S.-Y., Odono, R. M., & Hedman, T. P. (2007). Effects of exogenous crosslinking on in vitro tensile and compressive moduli of lumbar intervertebral discs. Clinical Biomechanics, 22(1), 14-20.
Counter, P., & Wilson, J. A. (2004). The management of simple snoring. Sleep Medicine Reviews, 8(6), 433-441. doi:10.1016/j.smrv.2004.03.007
Courey, M. S., Fomin, D., Smith, T., Huang, S., Sanders, D., & Reinisch, L. (1999). Histologic and physiologic effects of electrocautery, CO2 laser, and radiofrequency injury in the porcine soft palate. Laryngoscope, 109(8), 1316-1319. doi:10.1097/00005537-199908000-00025
Deegan, P. C., & McNicholas, W. T. (1995). PATHOPHYSIOLOGY OF OBSTRUCTIVE SLEEP-APNEA. European Respiratory Journal, 8(7), 1161-1178. doi:10.1183/09031936.95.08071161
Demir, A. U., Ardic, S., Firat, H., Karadeniz, D., Aksu, M., Ucar, Z. Z., . . . Comm, T. I. (2015). Prevalence of sleep disorders in the Turkish adult population epidemiology of sleep study. Sleep and Biological Rhythms, 13(4), 298-308. doi:10.1111/sbr.12118
Dyken, M. E., & Bin Im, K. (2009). Obstructive Sleep Apnea and Stroke. Chest, 136(6), 1668-1677. doi:10.1378/chest.08-1512
Evans, N. D., Oreffo, R. O. C., Healy, E., Thurner, P. J., & Man, Y. H. (2013). Epithelial mechanobiology, skin wound healing, and the stem cell niche*. Journal of the Mechanical Behavior of Biomedical Materials, 28, 397-409. doi:10.1016/j.jmbbm.2013.04.023
Ferini-Strambi, L., & Braghiroli, A. (2006). Epidemiology and social cost of obstructive sleep apnoea. Journal of Sleep Research, 15, 9-9. Retrieved from <Go to ISI>://WOS:000239966000019
Gratzer, P. F., & Lee, J. M. (2001). Control of pH alters the type of cross-linking produced by 1-ethyl-3-(3-dimethylaminopropyl)-carbodiimide (EDC) treatment of acellular matrix vascular grafts. Journal of Biomedical Materials Research, 58(2), 172-179. doi:10.1002/1097-4636(2001)58:2<172::aid-jbm1004>3.0.co;2-9
Gurtner, G. C., Werner, S., Barrandon, Y., & Longaker, M. T. (2008). Wound repair and regeneration. Nature, 453(7193), 314-321. doi:10.1038/nature07039
Han, B., Jaurequi, J., Tang, B. W., & Nimni, M. E. (2003). Proanthocyanidin: A natural crosslinking reagent for stabilizing collagen matrices. Journal of Biomedical Materials Research Part A, 65A(1), 118-124. doi:10.1002/jbm.a.10460
Hapach, L. A., VanderBurgh, J. A., Miller, J. P., & Reinhart-King, C. A. (2015). Manipulation of in vitro collagen matrix architecture for scaffolds of improved physiological relevance. Physical Biology, 12(6). doi:10.1088/1478-3975/12/6/061002
Hedman, T. P., Saito, H., Vo, C., & Chuang, S.-Y. (2006). Exogenous cross-linking increases the stability of spinal motion segments. Spine, 31(15), E480-E485. doi:10.1097/01.brs.0000224531.49174.ea
Hillman, D. R., Murphy, A. S., & Pezzullo, L. (2006). The economic cost of sleep disorders. Sleep, 29(3), 299-305.
Ho, W. K., Wei, W. I., & Chung, K. F. (2004). Managing disturbing snoring with palatal implants—A pilot study. Archives of Otolaryngology-Head & Neck Surgery, 130(6), 753-758. doi:10.1001/archoto1.130.6.753
Hoffmann, B., Seitz, D., Mencke, A., Kokott, A., & Ziegler, G. (2009). Glutaraldehyde and oxidised dextran as crosslinker reagents for chitosan-based scaffolds for cartilage tissue engineering. Journal of Materials Science-Materials in Medicine, 20(7), 1495-1503. doi:10.1007/s10856-009-3707-3
Hollinsky, C., & Sandberg, S. (2007). Measurement of the tensile strength of the ventral abdominal wall in comparison with scar tissue. Clinical Biomechanics, 22(1), 88-92. doi:10.1016/j.clinbiomech.2006.06.002
Ingman, T., Nieminen, T., & Hurmerinta, K. (2004). Cephalometric comparison of pharyngeal changes in subjects with upper airway resistance syndrome or obstructive sleep apnoea in upright and supine positions. Eur J Orthod, 26(3), 321-326. doi:10.1093/ejo/26.3.321
Ioachimescu, O. C., & Collop, N. A. Sleep-Disordered Breathing. Neurologic Clinics, 30(4), 1095-1136. doi:10.1016/j.nc1.2012.08.003
Jelovsek, J. E., Maher, C., & Barber, M. D. (2007). Pelvic organ prolapse. Lancet, 369(9566), 1027-1038. doi:10.1016/s0140-6736(07)60462-0
Jonn, J. Y., Bobo, J., Quintero, J., & Moseley, J. P. (2003). Absorbable adhesive compositions: Google Patents.
Jordan, A. S., McSharry, D. G., & Malhotra, A. (2014). Adult obstructive sleep apnoea. Lancet, 383(9918), 736-747. doi:10.1016/s0140-6736(13)60734-5
Klapperich, C. M., Noack, C. L., Kaufman, J. D., Zhu, L., Bonnaillie, L., & Wool, R. P. (2009). A novel biocompatible adhesive incorporating plant-derived monomers. Journal of Biomedical Materials Research Part A, 91A(2), 378-384. doi:10.1002/jbm.a.32250
Kotecha, B. T., & Hall, A. C. (2014). Role of surgery in adult obstructive sleep apnoea. Sleep Medicine Reviews, 18(5), 405-413. doi:10.1016/j.smrv.2014.02.003
Lau, Y.-K. I., Gobin, A. M., & West, J. L. (2006). Overexpression of lysyl oxidase to increase matrix crosslinking and improve tissue strength in dermal wound healing. Annals of Biomedical Engineering, 34(8), 1239-1246. doi:10.1007/s10439-006-9130-8
Mandavi, A., Ferreira, L., Sundback, C., Nichol, J. W., Chan, E. P., Carter, D. J. D., . . . Karp, J. M. (2008). A biodegradable and biocompatible gecko-inspired tissue adhesive. Proceedings of the National Academy of Sciences of the United States of America, 105(7), 2307-2312. doi:10.1073/pnas.0712117105
Marcussen, L., Henriksen, J. E., & Thygesen, T. (2015). Do Mandibular Advancement Devices Influence Patients' Snoring and Obstructive Sleep Apnea? A Cone-Beam Computed Tomography Analysis of the Upper Airway Volume. Journal of Oral and Maxillofacial Surgery, 73(9), 1816-1826. doi:10.1016/j.joms.2015.02.023
Neruntarat, C., & Chantapant, S. (2009). Radiofrequency surgery for the treatment of obstructive sleep apnea: Short-term and long-term results. Otolaryngology-Head and Neck Surgery, 141(6), 722-726. doi:10.1016/j.otohns.2009.09.028
Provenzano, P. P., Martinez, D. A., Grindeland, R. E., Dwyer, K. W., Turner, J., Vailas, A. C., & Vanderby, R. (2003). Hindlimb unloading alters ligament healing. Journal of Applied Physiology, 94(1), 314-324. doi:10.1152/japplphysiol.00340.2002
Ryan, C. M., & Bradley, T. D. (2005). Pathogenesis of obstructive sleep apnea. Journal of Applied Physiology, 99(6), 2440-2450. doi:10.1152/japplphysiol.00772.2005
Sadatsafavi, M., Marra, C. A., Ayas, N. T., Stradling, J., & Fleetham, J. (2009). Cost-effectiveness of oral appliances in the treatment of obstructive sleep apnoea-hypopnoea. Sleep and Breathing, 13(3), 241-252. doi:10.1007/s11325-009-0248-4
Schmack, I., Dawson, D. G., McCarey, B. E., Waring, G. O., Grossniklaus, H. E., & Edelhauser, H. F. (2005). Cohesive tensile strength of human LASIK wounds with histologic, ultrastructural, and clinical correlations. Journal of Refractive Surgery, 21(5), 433-445. Retrieved from <Go to ISI>://WOS:000232339000001
Singhal, A. R., Agrawal, C. M., & Athanasiou, K. A. (1996). Salient Degradation Features of a 50:50 PLA/PGA Scaffold for Tissue Engineering. Tissue engineering, 2(3), 197-207. doi:10.1089/ten.1996.2.197
Slusarewicz, P., Zhu, K., & Hedman, T. (2010). Kinetic characterization and comparison of various protein crosslinking reagents for matrix modification. Journal of Materials Science: Materials in Medicine, 21(4), 1175-1181.
Slusarewicz, P., Zhu, K., Kirking, B., Toungate, J., & Hedman, T. (2011). Optimization of protein crosslinking formulations for the treatment of degenerative disc disease. Spine, 36(1), E7.
Spicuzza, L., Caruso, D., & Di Maria, G. (2015). Obstructive sleep apnoea syndrome and its management. Therapeutic Advances in Chronic Disease, 6(5), 273-285. doi:10.1177/2040622315590318
Sung, H. W., Chang, Y., Chiu, C. T., Chen, C. N., & Liang, H. C. (1999a). Crosslinking characteristics and mechanical properties of a bovine pericardium fixed with a naturally occurring crosslinking agent. Journal of Biomedical Materials Research, 47(2), 116-126. doi:10.1002/(sici)1097-4636(199911)47:2<116::aid-jbm2>3.0.co;2-j
Sung, H. W., Chang, Y., Chiu, C. T., Chen, C. N., & Liang, H. C. (1999b). Mechanical properties of a porcine aortic valve fixed with a naturally occurring crosslinking agent. Biomaterials, 20(19), 1759-1772. doi:10.1016/s0142-9612(99)00069-1
Sung, H. W., Liang, I. L., Chen, C. N., Huang, R. N., & Liang, H. F. (2001). Stability of a biological tissue fixed with a naturally occurring crosslinking agent (genipin). Journal of Biomedical Materials Research, 55(4), 538-546. doi:10.1002/1097-4636(20010615)55:4<538::aid-jbm1047>3.0.co;2-2
Suratt, P. M., Dee, P., Atkinson, R. L., Armstrong, P., & Wilhoit, S. C. (1983). FLUOROSCOPIC AND COMPUTED TOMOGRAPHIC FEATURES OF THE PHARYNGEAL AIRWAY IN OBSTRUCTIVE SLEEP-APNEA. American Review of Respiratory Disease, 127(4), 487-482. Retrieved from <Go to ISI>://WOS:A1983QL66100019
Sutherland, K., & Cistulli, P. A. (2015). Recent advances in obstructive sleep apnea pathophysiology and treatment. Sleep and Biological Rhythms, 13(1), 26-40. doi:10.1111/sbr.12098
Tang, S. Y., Sharan, A. D., & Vashishth, D. (2008). Effects of collagen crosslinking on tissue fragility. Clinical Biomechanics, 23(1), 122-123. doi:10.1016/j.clinbiomech.2007.08.010
Vasudev, S. C., & Chandy, T. (1997). Effect of alternative crosslinking techniques on the enzymatic degradation of bovine pericardia and their calcification. Journal of Biomedical Materials Research, 35(3), 357-369. doi:10.1002/(sici)1097-4636(19970605)35:3<357::aid-jbm10>3.0.co;2-c
Verzijl, N., DeGroot, J., Ben Zaken, C., Braun-Benjamin, O., Maroudas, A., Bank, R. A., . . . TeKoppele, J. M. (2002). Crosslinking by advanced glycation end products increases the stiffness of the collagen network in human articular cartilage—A possible mechanism through which age is a risk factor for osteoarthritis. Arthritis and Rheumatism, 46(1), 114-123. doi:10.1002/1529-0131(200201)46:1<114::aid-art10025>3.0.co;2-p
Wagner, D. R., Reiser, K. M., & Lotz, J. C. (2006). Glycation increases human annulus fibrosus stiffness in both experimental measurements and theoretical predictions. Journal of Biomechanics, 39(6), 1021-1029. doi:10.1016/j.jbiomech.2005.02.013
Wu, X., Black, L., Santacana-Laffitte, G., & Patrick, C. W., Jr. (2007). Preparation and assessment of glutaraldehyde-crosslinked collagen-chitosan hydrogels for adipose tissue engineering. Journal of Biomedical Materials Research Part A, 81A(1), 59-65. doi:10.1002/jbm.a.31003
Yang, S. H., Hsu, C. K., Wang, K. C., Hou, S. M., & Lin, F. H. (2005). Tricalcium phosphate and glutaraidehyde crosslinked gelatin incorporating bone morphogenetic protein—A viable scaffold for bone tissue engineering. Journal of Biomedical Materials Research Part B-Applied Biomaterials, 74B(1), 468-475. doi:10.1002/jbm.b.30200
Yerramalli, C. S., Chou, A. I., Miller, G. J., Nicoll, S. B., Chin, K. R., & Elliott, D. M. (2007). The effect of nucleus pulposus crosslinking and glycosaminoglycan degradation on disc mechanical function. Biomechanics and Modeling in Mechanobiology, 6(1-2), 13-20. doi:10.1007/s10237-006-0043-0
Young, T., Palta, M., Dempsey, J., Skatrud, J., Weber, S., & Badr, S. (1993). The Occurrence of Sleep-Disordered Breathing among Middle-Aged Adults. New England Journal of Medicine, 328(17), 1230-1235. doi:doi:10.1056/NEJM199304293281704
Zhai, W. Y., Chang, J., Lin, K. L., Wang, J. Y., Zhao, Q., & Sun, X. N. (2006). Crosslinking of decellularized porcine heart valve matrix by procyanidins. Biomaterials, 27(19), 3684-3690. doi:10.1016/j.biomaterials.2006.02.008

1: A method for improving fibrotic tissue in a subject comprising:
a. performing a stiffening procedure on a soft tissue of a subject to induce a fibrotic tissue within the soft tissue; and
b. administering in vivo a chemical crosslinking reagent comprising a crosslinker to the fibrotic tissue.

2: The method of claim 1, wherein the chemical crosslinking reagent is administered between 3 days and 3 weeks following the stiffening procedure.

3: The method of claim 2, wherein the chemical crosslinking reagent is administered 3 to 10 days after the stiffening procedure.

4: The method of claim 1, wherein the chemical crosslinking reagent is administered as a solution, spray, gel, encapsulation, or released from a device, coating, patch, strip, suture, implant or combination thereof.

5: The method of claim 1, wherein the chemical crosslinking reagent comprises a crosslinker and a carrier solution.

6: The method of claim 1, wherein the crosslinker is selected from the group consisting of D- or L-Threose, genipin (GP), methylglyoxal (MG), 1-ethyl-3-(3-dimethylamniopropyl) carbodiimide hydrochloride (EDC), proanthrocyanidin, transglutaminase (TG) or combinations thereof.

7: The method of claim 5, wherein the crosslinker is 20-100 mM of D- and/or L-Threose.

8: The method of claim 5, wherein the crosslinker is 5-120 mM of genipin.

9: The method of claim 5, wherein the crosslinker is 5-50 mM of methylglyoxal.

10: The method of claim 5, wherein the crosslinker is 2-50 mM of 1-ethyl-3-(3-dimethylamniopropyl) carbodilimide hydrochloride.

11: The method of claim 5, wherein the crosslinker is 0.025-0.5% w/v of proanthrocyanidin.

12: The method of claim 5, wherein the crosslinker is 0.5-5 U/ml of transglutaminase.

13: The method of claim 1, wherein the carrier solution comprises an additive selected from the group consisting of a buffer, a surfactant, a stabilization agent, a co-factor, a flavoring agent or combinations thereof.

14: The method of claim 1, wherein the chemical crosslinking reagent further comprises a biodegradable or non-degradable polymer to control the release of the crosslinker.

15: The method of claim 1, wherein the stiffening procedure is selected from the group consisting of radio-frequency (RF) heating, radio-frequency ablation (RFA), administration of laser energy, ethanol administration, hypertonic saline administration, sodium tetradecyl sulphate administration, doxycycline administration, insertion of palatal implants or combinations thereof.

16: A method for improving fibrotic tissue in a subject comprising the steps of: preparing a crosslinking reagent by dissolving a crosslinking agent in a carrier medium; and contacting at least a portion of the fibrotic tissue in a subject.

17: The method of claim 16, wherein the contact between the fibrotic tissue and the crosslinking reagent is affected by injections directly into the portion of the fibrotic tissue with a needle.

18: A composition for inducing crosslinking in fibrotic tissue comprising a chemical crosslinking reagent comprising a crosslinker and a carrier solution.

19: The composition of claim 18, wherein the crosslinker comprises 50 mM of genipin.

20: The composition of claim 19, wherein the carrier solution comprises water.

21: The composition of claim 20, wherein the carrier solution further comprises isotonic factors such as phosphate.

22: The method of claim 14, wherein the crosslinker is distributed throughout the biodegradable or non-degradable polymer at a ratio of about 5-100 mg crosslinker to 1 g of polymer.

23: The method of claim 14, wherein the crosslinker comprises genipin and the genipin is distributed throughout the biodegradable or non-degradable polymer at a ratio of about 10 mg genipin to 1 g of polymer.

24: The method of claim 14, wherein the chemical crosslinking reagent is prevented from release from a biodegradable or non-degradable polymer until after an outer coating comprised of a biodegradable polymer has degraded.
